(19) United States
(12) Patent Application Publication
(10) Pub. No.: US 2019/0295248 A1
(43) Pub. Date: Sep. 26, 2019

(54) MEDICAL IMAGE SPECIFYING APPARATUS, METHOD, AND PROGRAM

(71) Applicant: FUJIFILM Corporation, Tokyo (JP)

(72) Inventors: Keigo NAKAMURA, Tokyo (JP); Yohei MOMOKI, Kanagawa (JP)

(73) Assignee: FUJIFILM Corporation, Tokyo (JP)

(21) Appl. No.: 16/279,281

(22) Filed: Feb. 19, 2019

(30) Foreign Application Priority Data
Mar. 26, 2018 (JP) 2018-057770

(51) Int. Cl. G06T 7/00; G16H 30/40

(52) U.S. Cl. CPC G06T 7/0012; G16H 50/20; G16H 30/40

(57) ABSTRACT

A character information acquisition unit acquires character information relevant to a medical image group including a plurality of medical images. An image character information conversion unit converts each of the plurality of medical images into image character information corresponding to each medical image. A specifying unit specifies at least one relevant medical image, which is relevant to an interpretation report that is character information, from the plurality of medical images by comparing the image character information corresponding to each of the plurality of medical images with the interpretation report.

### CROSS REFERENCE TO RELATED APPLICATIONS

The present application claims priority under 35 U.S.C. § 119 to Japanese Patent Application No. 2018-057770 filed on Mar. 26, 2018. The above application is hereby expressly incorporated by reference, in its entirety, into the present application.

### BACKGROUND

### Field of the Invention

The present invention relates to a medical image specifying apparatus, method, and program for specifying a medical image relevant to a medical document, such as an interpretation report, from a plurality of medical images.

### Related Art

In recent years, advances in medical apparatuses, such as computed tomography (CT) apparatuses and magnetic resonance imaging (MRI) apparatuses, have enabled image diagnosis using high-resolution three-dimensional medical images with higher quality. In particular, since the region of a lesion can be accurately specified by image diagnosis using CT images, MRI images, and the like, appropriate treatment can be performed based on the specified result.

A medical image is analyzed by computer aided diagnosis (CAD) using a discriminator learned by deep learning or the like, regions, positions, volumes, and the like of lesions included in the medical image are extracted, and these are acquired as the analysis result. The analysis result generated by analysis processing in this manner is stored in a database so as to be associated with examination information, such as a patient name, gender, age, and a modality that has acquired the medical image, and provided for diagnosis. At this time, a technician in a radiology department or the like, who has acquired the medical image, determines a radiologist according to the medical image, and notifies the determined radiologist that the medical image and the result of analysis by the CAD are present. The radiologist interprets the medical image with reference to the transmitted medical image and analysis result and creates an interpretation report at his or her own interpretation terminal.

An interpretation report on a three-dimensional medical image is stored in an image server or the like so as to be associated with a medical image referred to at the time of creating the interpretation report among a plurality of two-dimensional medical images forming the three-dimensional medical image. Therefore, referring to the interpretation report, it is easy to specify the medical image referred to at the time of creating the interpretation report and display the medical image on the terminal device of the doctor. However, an interpretation report associated with a medical image whose imaging time is old may not be associated with a medical image referred to at the time of creating the interpretation report on the system. In such a case, it is difficult to specify and display a medical image referred to at the time of creating the interpretation report. In particular, since a three-dimensional medical image, such as a CT image and an MRI image, is configured to include a number of tomographic images, it is very difficult to specify which tomographic image has been referred to in order to create the interpretation report.

For this reason, various methods have been proposed to associate a medical image and character information included in a medical document, such as an interpretation report, with each other. For example, a method has been proposed that specifies a tomographic image accurately expressing disease information by using diagnostic information created for a three-dimensional image including a plurality of series of tomographic images (refer to JP2013-233470A). In addition, a method has been proposed that acquires information including a finding item of interest, among a plurality of finding items, and the value of the finding for a three-dimensional image to be examined, calculates a feature amount corresponding to the finding item of interest from each of a plurality of tomographic images forming the three-dimensional image, and specifies a tomographic image based on the calculated feature amount and the value of the finding (refer to JP2014-000351A).

In the method disclosed in JP2013-233470A, however, character information included in diagnostic information is converted into information corresponding to an image. In the method disclosed in JP2014-000351A, the feature amount corresponding to the finding item of interest is calculated from each of the plurality of tomographic images forming a three-dimensional image. For this reason, in the methods disclosed in JP2013-233470A and JP2014-000351A, the amount of calculation for specifying a tomographic image corresponding to character information included in a medical document, such as an interpretation report, is increased, and the processing requires time.

### SUMMARY

The invention has been made in view of the above circumstances, and it is an object of the invention to make it possible to quickly specify a medical image, which is relevant to character information included in a medical document, from a medical image group including a plurality of medical images.

A medical image specifying apparatus according to the invention comprises: a character information acquisition unit that acquires character information relevant to a medical image group including a plurality of medical images; an image character information conversion unit that converts each of the plurality of medical images into image character information corresponding to each medical image; and a specifying unit that specifies at least one relevant medical image, which is relevant to the character information, from the plurality of medical images by comparing the character information with the image character information corresponding to each of the plurality of medical images.

“Character information” is information of characters indicating the features of a lesion or the like included in medical documents, such as an interpretation report, a diagnosis report, and an electronic medical record. Specifically, information indicating the features of a lesion, such as the type of a lesion included in a medical document, the position of the lesion, the size of the lesion, can be used as character information.

In the medical image specifying apparatus according to the invention, the image character information conversion unit may comprise: a region extraction unit that extracts a region of at least one structure included in each of the medical images; and an analysis unit that analyzes the extracted region of the structure and expresses features of the region of the structure in text.

In this case, the features of the region of the structure may include at least one of a position of a lesion included in the structure, a type of the lesion, or a size of the lesion.

The “structure” means an organ, a muscle, a skeleton, and the like forming a human body included in a medical image. For example, a medical image of the chest includes the heart, the lung, muscles, bones, and the like, each of which is a structure.

In the medical image specifying apparatus according to the invention, the specifying unit may further specify a position relevant to the character information in the relevant medical image.

The medical image specifying apparatus according to the invention may further comprise a display control unit that displays the specified relevant medical image and the character information on a display unit.

In the medical image specifying apparatus according to the invention, the display control unit may highlight a position relevant to the character information in the relevant medical image.

In the medical image specifying apparatus according to the invention, the medical image may be a tomographic image, and the medical image group may be a three-dimensional image including a plurality of the tomographic images.

A medical image specifying method according to the invention comprises: acquiring character information relevant to a medical image group including a plurality of medical images; converting each of the plurality of medical images into image character information corresponding to each medical image; and specifying at least one relevant medical image, which is relevant to the character information, from the plurality of medical images by comparing the character information with the image character information corresponding to each of the plurality of medical images.

In addition, a program causing a computer to execute the medical image specifying method according to the invention may be provided.

Another medical image specifying apparatus according to the invention comprises: a memory that stores commands to be executed by a computer; and a processor configured to execute the stored commands. The processor executes: processing for acquiring character information relevant to a medical image group including a plurality of medical images; processing for converting each of the plurality of medical images into image character information corresponding to each medical image; and processing for specifying at least one relevant medical image, which is relevant to the character information, from the plurality of medical images by comparing the character information with the image character information corresponding to each of the plurality of medical images.

According to the invention, character information relevant to a medical image group including a plurality of medical images is acquired, each of the plurality of medical images is converted into image character information corresponding to each medical image, and at least one relevant medical image is specified from the plurality of medical images by comparing the character information with the image character information corresponding to each of the plurality of medical images. Here, the processing for converting an image into characters can be performed at a higher speed than the processing for converting characters into an image. Therefore, according to the invention, a relevant medical image relevant to the character information can be specified at high speed.

### BRIEF DESCRIPTION OF THE DRAWINGS

FIG. 1 is a diagram showing the schematic configuration of a medical information system to which a medical image specifying apparatus according to an embodiment of the invention is applied.

FIG. 2 is a diagram showing the schematic configuration of the medical image specifying apparatus according to the present embodiment.

FIG. 3 is a schematic block diagram showing the configuration of an image character information conversion unit.

FIG. 4 is a diagram illustrating the specification of a relevant tomographic image.

FIG. 5 is a diagram showing an interpretation report screen.

FIG. 6 is a flowchart showing a process performed in the present embodiment.

FIG. 7 is a flowchart of an image character information conversion process.

### DETAILED DESCRIPTION

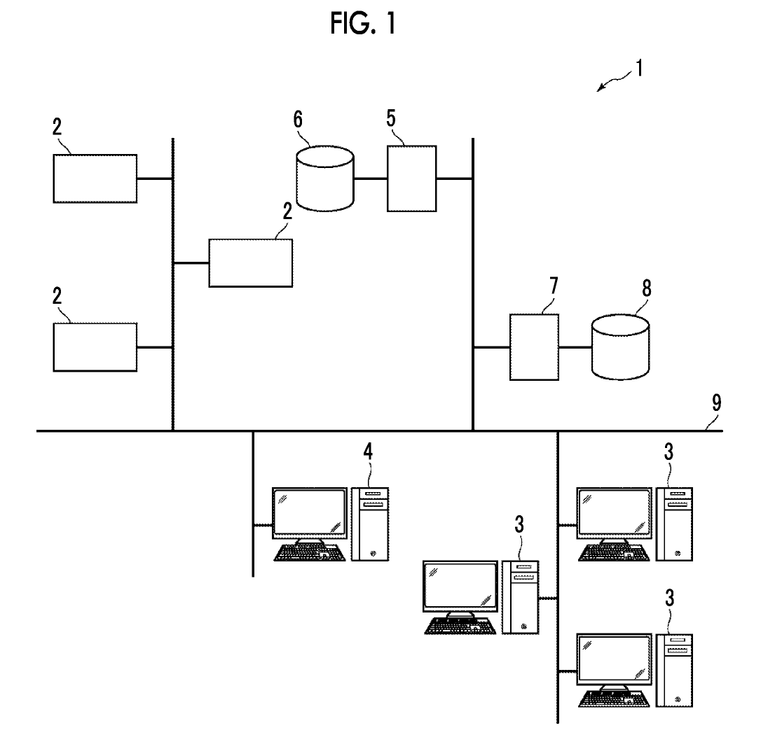

Hereinafter, an embodiment of the invention will be described with reference to the accompanying diagrams. FIG. 1 is a diagram showing the schematic configuration of a medical information system to which a medical image specifying apparatus according to an embodiment of the invention is applied. A medical information system 1 shown in FIG. 1 is a system for performing imaging of an examination target part of a subject, storage of a medical image acquired by imaging, interpretation of a medical image by a radiologist and creation of an interpretation report, and viewing of an interpretation report by a doctor in a medical department of a request source and detailed observation of a medical image to be interpreted, based on an examination order from a doctor in a medical department using a known ordering system. As shown in FIG. 1, the medical information system 1 is configured to include a plurality of modalities (imaging apparatuses) 2, a plurality of interpretation workstations (WS) 3 that are interpretation terminals, a medical department workstation (WS) 4, an image server 5, an image database 6, an interpretation report server 7, and an interpretation report database 8 that are communicably connected to each other through a wired or wireless network 9.

Each apparatus is a computer on which an application program for causing each apparatus to function as a component of the medical information system 1 is installed. The application program is recorded on a recording medium, such as a digital versatile disc (DVD) or a compact disc read only memory (CD-ROM), and distributed, and is installed onto the computer from the recording medium. Alternatively, the application program is stored in a storage device of a server computer connected to the network 9 or in a network storage so as to be accessible from the outside, and is downloaded and installed onto the computer as necessary.

The modality 2 is an apparatus that generates a medical image showing a diagnosis target part by imaging the diagnosis target part of the subject. Specifically, the modality 2 is a simple X-rays imaging apparatus, a CT apparatus, an MRI apparatus, a positron emission tomography (PET) apparatus, and the like. A medical image generated by the modality 2 is transmitted to the image server 5 and stored therein.

The interpretation WS 3 includes the medical image specifying apparatus according to the present embodiment. The configuration of the interpretation WS 3 will be described later.

The medical department WS 4 is a computer used by a doctor in a medical department to observe the details of an image, view an interpretation report, create an electronic medical record, and the like, and is configured to include a processing device, a display device such as a display, and an input device such as a keyboard and a mouse. In the medical department WS 4, each process, such as creation of a patient's medical record (electronic medical record), sending a request to view an image to the image server 5, display of an image received from the image server 5, automatic detection or highlighting of a lesion-like portion in an image, sending a request to view an interpretation report to the interpretation report server 7, and display of an interpretation report received from the interpretation report server 7, is performed by executing a software program for each process.

The image server 5 is obtained by installing a software program for providing a function of a database management system (DBMS) on a general-purpose computer. The image server 5 comprises a storage for an image database 6. This storage may be a hard disk device connected to the image server 5 by a data bus, or may be a disk device connected to a storage area network (SAN) or a network attached storage (NAS) connected to the network 9. In a case where the image server 5 receives a request to register a medical image from the modality 2, the image server 5 registers the medical image in the image database 6 in a format for a database.

Medical images acquired by the modality 2 or image data of a medical image group including a plurality of medical images and accessory information are registered in the image database 6. The accessory information includes, for example, an image ID for identifying each medical image or a medical image group (hereinafter, may be simply referred to as a medical image), a patient identification (ID) for identifying a subject, an examination ID for identifying an examination, a unique ID (UID: unique identification) allocated for each medical image, examination date and examination time at which the medical image or the medical image group is generated, the type of a modality used in an examination for acquiring a medical image, patient information such as patient's name, age, and gender, an examination part (imaging part), imaging information (an imaging protocol, an imaging sequence, an imaging method, imaging conditions, the use of a contrast medium, and the like), and information such as a series number or a collection number in a case where a plurality of medical images are acquired in one examination.

In a case where a viewing request from the interpretation WS 3 is received through the network 9, the image server 5 searches for a medical image registered in the image database 6 and transmits the searched medical image to the interpretation WS 3 that is a request source.

The interpretation report server 7 has a software program for providing a function of a database management system to a general-purpose computer. In a case where the interpretation report server 7 receives a request to register an interpretation report from the interpretation WS 3, the interpretation report server 7 registers the interpretation report in the interpretation report database 8 in a format for a database. In a case where a request to search for an interpretation report is received, the interpretation report is searched for from the interpretation report database 8.

In the interpretation report database 8, for example, an interpretation report is registered in which information, such as an image ID for identifying a medical image to be interpreted, a radiologist ID for identifying an image diagnostician who performed the interpretation, a lesion name, position information of a lesion, findings, and the certainty factor of findings, is recorded.

The network 9 is a wired or wireless local area network that connects various apparatuses in a hospital to each other. In a case where the interpretation WS 3 is installed in another hospital or clinic, the network 9 may be configured to connect local area networks of respective hospitals through the Internet or a dedicated circuit. In any case, it is preferable that the network 9 is configured to be able to realize high-speed transmission of medical images, such as an optical network.

Hereinafter, the interpretation WS 3 according to the present embodiment will be described in detail. The interpretation WS 3 is a computer used by a radiologist of a medical image to interpret the medical image and create the interpretation report, and is configured to include a processing device, a display device such as a display, and an input device such as a keyboard and a mouse. In the interpretation WS 3, each process, such as making a request to view a medical image to the image server 5, various kinds of image processing on a medical image received from image server 5, display of a medical image, analysis processing on a medical image, highlighting of a medical image based on the analysis result, creation of an interpretation report based on the analysis result, support for the creation of an interpretation report, making a request to register an interpretation report and a request to view an interpretation report to the interpretation report server 7, and display of an interpretation report received from the interpretation report server 7, is performed by executing a software program for each process. Since processes other than the process performed by the medical image specifying apparatus according to the present embodiment, among these processes, are performed by a known software program, the detailed description thereof will be omitted herein. The processes other than the process performed by the medical image specifying apparatus according to the present embodiment may not be performed in the interpretation WS 3, and a computer that performs the processes may be separately connected to the network 9, and requested processing on the computer may be performed according to a processing request from the interpretation WS 3.

The interpretation WS 3 includes the medical image specifying apparatus according to the present embodiment. Therefore, a medical image specifying program according to the present embodiment is installed on the interpretation WS 3. The medical image specifying program is recorded on a recording medium, such as a DVD or a CD-ROM, and distributed, and is installed onto the interpretation WS 3 from the recording medium. Alternatively, the medical image specifying program is stored in a storage device of a server computer connected to the network or in a network storage so as to be accessible from the outside, and is downloaded and installed onto the interpretation WS 3 as necessary.

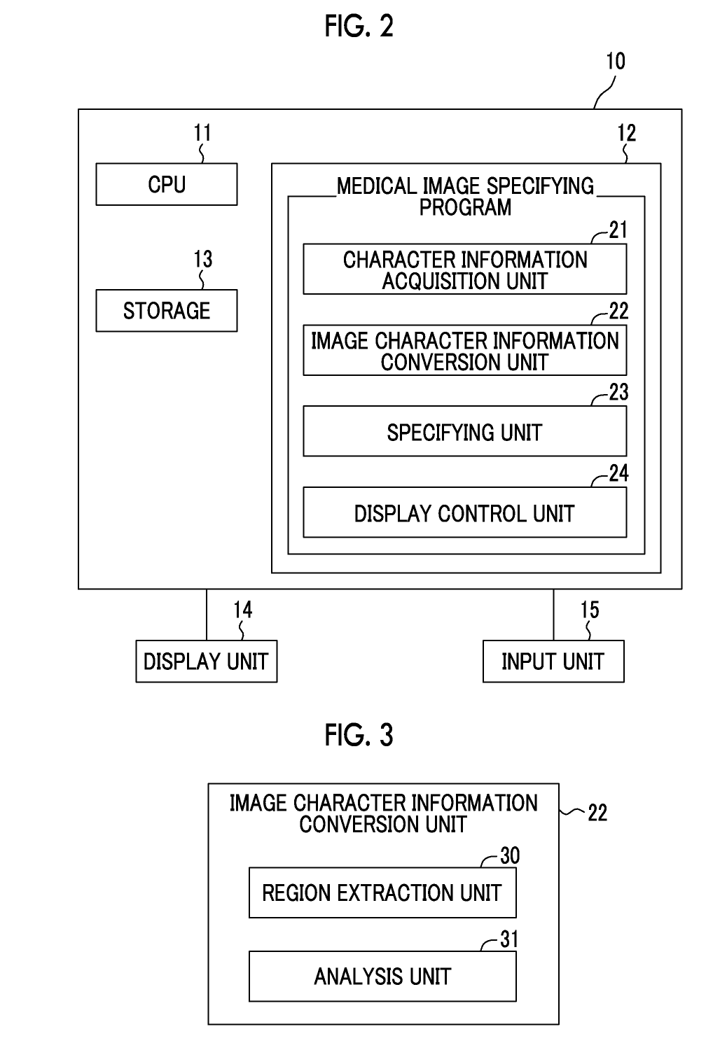

FIG. 2 is a diagram showing the schematic configuration of the medical image specifying apparatus according to the embodiment of the invention that is realized by installing the medical image specifying program. As shown in FIG. 2, a medical image specifying apparatus 10 comprises a central processing unit (CPU) 11, a memory 12, and a storage 13 as the configuration of a standard computer. A display device (hereinafter, referred to as a display unit) 14, such as a liquid crystal display, and an input device (hereinafter, referred to as an input unit) 15, such as a keyboard and a mouse, are connected to the medical image specifying apparatus 10.

The storage 13 is a storage device, such as a hard disk or a solid state drive (SSD). Medical images and various kinds of information including information necessary for processing of the medical image specifying apparatus 10, which are acquired from the image server 5 through the network 9, are stored in the storage 13.

A medical image specifying program is stored in the memory 12. As processing to be executed by the CPU 11, the medical image specifying program defines: character information acquisition processing for acquiring character information relevant to a medical image group including a plurality of medical images; image character information conversion processing for converting each of the plurality of medical images into image character information corresponding to each medical image; specifying processing for specifying at least one relevant medical image, which is relevant to the character information, from the plurality of medical images by comparing the character information with the image character information corresponding to each of the plurality of medical images; and display control processing for displaying the specified relevant medical image and the character information on a display unit 14.

The CPU 11 executes these processes according to the medical image specifying program, so that the computer functions as a character information acquisition unit 21, an image character information conversion unit 22, a specifying unit 23, and a display control unit 24. In the present embodiment, the CPU 11 executes the function of each unit according to the medical image specifying program. However, as a general-purpose processor that executes software to function as various processing units, a programmable logic device (PLD) that is a processor whose circuit configuration can be changed after manufacturing, such as a field programmable gate array (FPGA), can be used in addition to the CPU 11. Alternatively, the processing of each unit may also be executed by a dedicated electric circuit that is a processor having a circuit configuration designed exclusively to execute specific processing, such as an application specific integrated circuit (ASIC).

One processing unit may be configured by one of various processors, or may be a combination of two or more processors of the same type or different types (for example, a combination of a plurality of FPGAs or a combination of a CPU and an FPGA). Alternatively, a plurality of processing units may be configured by one processor. As an example of configuring a plurality of processing units using one processor, first, as represented by a computer, such as a client and a server, there is a form in that one processor is configured by a combination of one or more CPUs and software and this processor functions as a plurality of processing units. Second, as represented by a system on chip (SoC) or the like, there is a form of using a processor that realizes the function of the entire system including a plurality of processing units with one integrated circuit (IC) chip. Thus, various processing units are configured by using one or more of the above-described various processors as a hardware structure.

More specifically, the hardware structure of these various processors is an electrical circuit (circuitry) in the form of a combination of circuit elements, such as semiconductor elements.

The character information acquisition unit 21 acquires character information relevant to a medical image group including a plurality of medical images. In the present embodiment, a three-dimensional image corresponds to the medical image group, and a plurality of tomographic images forming a three-dimensional image correspond to the medical image. In the present embodiment, it is assumed that the three-dimensional image is a CT image of a thoracoabdominal portion.

In order to acquire character information, the character information acquisition unit 21 instructs the interpretation report server 7 to send an interpretation report created for the three-dimensional image to be processed. The interpretation report server 7 acquires the requested interpretation report from the interpretation report database 8 and transmits the acquired interpretation report to the interpretation WS 3. The character information acquisition unit 21 extracts information indicating the features of a lesion, such as the position, type, and size of a lesion included in the interpretation report, as report character information by analyzing the character string included in the interpretation report transmitted from the interpretation report server 7 using the technique of natural language processing.

The natural language processing is a series of techniques for making a computer process the natural language used by humans on a daily basis. By natural language processing, division of a sentence into words, analysis of syntax, analysis of a meaning, and the like can be performed. In the present embodiment, the character information acquisition unit 21 acquires report character information by dividing the character string included in the interpretation report into words and analyzing the syntax using the technique of natural language processing. For example, in a case where the sentence of the interpretation report is “A nodule having a size of 25×21 mm is recognized in the upper lobe of the right lung.”, the character information acquisition unit 21 acquires the terms “right lung”, “upper lobe”, “nodule”, and “25×21 mm” as report character information.

A table in which terms, such as types of lesions and positions of lesions, are registered may be stored in the storage 13, and a character string matching the term may be extracted as report character information from the interpretation report with reference to the table. The report character information corresponds to character information.

Here, the image server 5 acquires the three-dimensional image to be processed from the image database 6 in response to a request from the interpretation WS 3, and transmits the acquired three-dimensional image to the interpretation WS 3. The three-dimensional image and the interpretation report are stored in the storage 13.

The three-dimensional image and the interpretation report are associated with each other. Accordingly, from one of the three-dimensional image or the interpretation report, the other one can be specified. On the other hand, it is assumed that each tomographic image forming the three-dimensional image is not associated with the interpretation report. That is, it is assumed that a link to each tomographic image forming the three-dimensional image, for which an interpretation report is to be created, is given to the interpretation report.

The image character information conversion unit 22 converts each of the plurality of medical images into image character information corresponding to each medical image. Specifically, each of the plurality of tomographic images forming the three-dimensional image is converted into character image information. FIG. 3 is a schematic block diagram showing the configuration of the image character information conversion unit. As shown in FIG. 3, the image character information conversion unit 22 includes a region extraction unit 30 and an analysis unit 31.

The region extraction unit 30 extracts a region of at least one structure included in each medical image. Here, structures included in the thoracoabdominal portion of the human body are lung field, heart, liver, muscle, fat, bone, and the like. The region extraction unit 30 extracts regions of structures, such as a lung field region, a heart region, a liver region, and a bone region, from each of a plurality of tomographic images. The region extraction unit 30 extracts a region of each structure from each tomographic image using a discriminator. The discriminator is machine-learned using a method, such as deep learning and AdaBoost, so that a tomographic image is input and an extracted structure is output. The region extraction unit 30 inputs each of the plurality of tomographic images to the discriminator and acquires the region of the output structure, thereby extracting the regions of the plurality of structures from the tomographic images.

As a method of extracting a structure from a tomographic image, in addition to a method using a discriminator, threshold processing based on the CT value of a tomographic image, a region growing method based on a seed point representing a structure, a template matching method based on the shape of a structure, a graph cutting method described in, for example, JP4493679B, and the like can be used.

The analysis unit 31 analyzes the region of a structure and expresses the features of the region of the structure in text. First, the analysis unit 31 analyzes the region of the structure extracted from the tomographic image, and acquires the analysis result on the disease and the like included in each tomographic image. For this purpose, the analysis unit 31 comprises a discriminator that is machine-learned to determine whether or not each pixel in the region of the structure indicates a lesion and to determine the type of a lesion in a case where the pixel is a lesion. In the present embodiment, the discriminator is a neural network deep-learned so as to be able to classify a plurality of types of lesions included in the image of each structure for every type of structure. In a case where the image of the region of a structure is input, the discriminator in the analysis unit 31 determines which of a plurality of types of lesions each pixel in the input image is, and outputs the determination result. In addition, it is assumed that the determination result includes not having any lesion.

The discriminator provided in the region extraction unit 30 and the analysis unit 31 may be configured to include, for example, a support vector machine (SVM), a convolutional neural network (CNN), and a recurrent neural network (RNN), in addition to the deep-learned neural network.

The analysis unit 31 expresses the features of the region of each structure in text using the discrimination result of the discriminator described above. For example, in a case where the region of the structure is a lung field region and a lesion is found in the lung field as a result of the analysis, the analysis unit 31 acquires at least one of the position of the lesion within the lung field, the type of the lesion, or the size of the lesion as a feature of the lung field region and expresses the acquired one in text. It is assumed that the size of the lesion is expressed by the size (length×width) of the lesion. Specifically, the analysis unit 31 calculates the size of the lesion by counting the number of pixels in the longitudinal direction and the horizontal direction of the lesion and multiplying the counted number of pixels by the size per pixel.

Here, in the present embodiment, it is assumed that all of the position of the lesion, the type of the lesion, and the size of the lesion are acquired. Specifically, as a result of analysis, in a case where a large nodule having a size of 25×21 mm is detected as a feature in the upper lobe of the right lung, the analysis unit 31 expresses the acquired feature in text as “right lung” and “upper lobe” indicating, the position of the lesion, “nodule” indicating the type of the lesion, and “25×21 mm” indicating the size of the lesion. Then, the analysis unit 31 outputs the information expressed in text described above as image character information regarding the medical image from which the analyzed region of the structure has been extracted. In a case where no lesion is detected from all structures included in each tomographic image, the analysis unit 31 outputs “none” as image character information for the tomographic image.

The specifying unit 23 specifies at least one relevant tomographic image, which is relevant to the interpretation report, from the plurality of tomographic images by comparing the image character information corresponding to each of the plurality of tomographic images with the report character information acquired by the character information acquisition unit 21. In the present embodiment, it is assumed that the specifying unit 23 specifies one relevant tomographic image from the plurality of tomographic images.

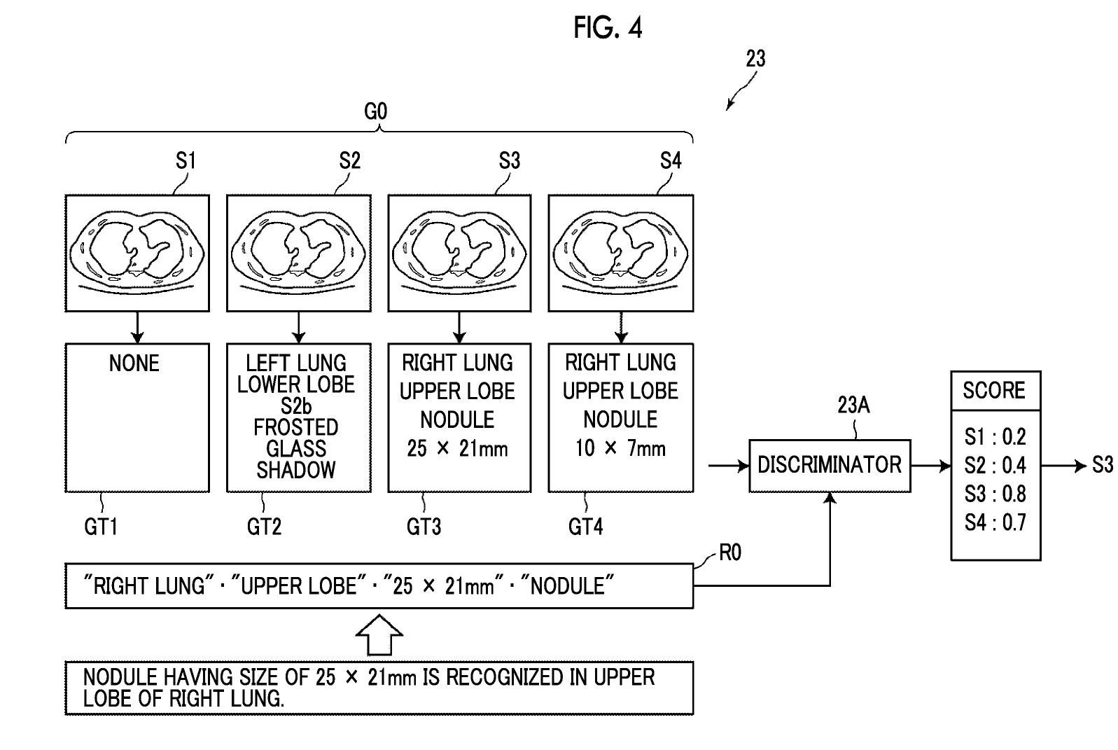

The specifying unit 23 specifies a relevant tomographic image by determining whether the image character information acquired from each of the plurality of tomographic images matches the interpretation report character information. FIG. 4 is a diagram illustrating the specification of a relevant tomographic image. In the present embodiment, it is assumed that a three-dimensional image GO is formed by four tomographic images S1 to S4. Then, it is assumed that the tomographic image S1 is converted into image character information GT1 of “none” by the image character information conversion unit 22 and the tomographic image S2 is converted into image character information GT2 of “left lung”, “lower lobe”, “S2b”, and “frosted glass shadow” by the image character information conversion unit 22. In addition, it is assumed that the tomographic image S3 is converted into image character information GT3 of “right lung”, “upper lobe”, “nodule”, and “25×21 mm” by the image character information conversion unit 22, and the tomographic image S4 is converted into image character information GT4 of “right lung”, “upper lobe”, “nodule”, and “10×7 mm” by the image character information conversion unit 22. In addition, it is assumed that report character information R0 of “right lung”, “upper lobe”, “25×21 mm”, and “nodule” is acquired from the interpretation report of “A nodule having a size of 25×21 mm is recognized in the upper lobe of the right lung.” by the character information acquisition unit 21.

In the present embodiment, the specifying unit 23 has a discriminator 23A that is a neural network deep-learned so as to output a score indicating the degree of matching between the input image character information GT1 to GT4 and the report character information R0. Then, the specifying unit 23 specifies a tomographic image having the highest score, among the tomographic images S1 to S4 whose scores output from the discriminator 23A exceed a predetermined threshold value Th1, as one relevant tomographic image that is relevant to the interpretation report. For example, assuming that the threshold value Th1 is 0.6, in a case where the scores of the tomographic images S1 to S4 are 0.2, 0.4, 0.8, and 0.7, the tomographic image S3 is specified as a relevant tomographic image.

The specifying unit 23 associates the relevant tomographic image with the interpretation report. Specifically, for one of the relevant tomographic image and the interpretation report, a link to the other one is given. For example, a link to the tomographic image S3 is given to the interpretation report. The specifying unit 23 further specifies a position relevant to the report character information in the relevant tomographic image based on the report character information R0 and the analysis result of the analysis unit 31 in the image character information conversion unit 22. For example, the position of the nodule in the tomographic image S3 is associated with the report character information of “nodule”.

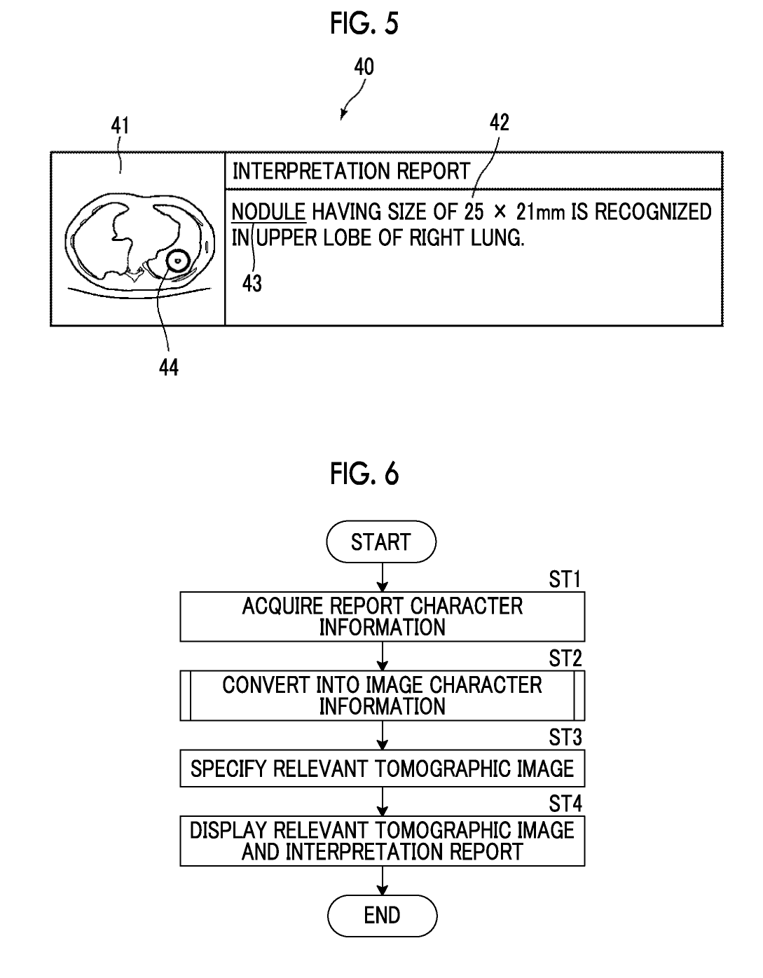

The display control unit 24 displays an interpretation report screen including the specified tomographic image and the interpretation report on the display unit 14. FIG. 5 is a diagram showing an interpretation report screen. As shown in FIG. 5, on an interpretation report screen 40, a relevant tomographic image 41 (for example, the tomographic image S3) specified by the specifying unit 23 is displayed, and a display region 42 of the interpretation report is displayed on the right side of the relevant tomographic image 41. In the display region 42, a sentence of “A nodule having a size of 25×21 mm is recognized in the upper lobe of the right lung.”, which is the interpretation report, is displayed. Underline is given to the character of “nodule” in this sentence, and a link 43 to the position of the nodule included in the relevant tomographic image 41 is generated. Here, in a case where the operator selects the link 43 using the input unit 15, the display control unit 24 highlights the location of the nodule in the relevant tomographic image 41. In FIG. 5, highlighting is indicated by adding a circle 44.

Next, a process performed in the present embodiment will be described. FIG. 6 is a flowchart showing the process performed in the present embodiment. In a case where a three-dimensional image to be processed and an interpretation report are transmitted to the interpretation WS 3 and an instruction to perform the process is given by the operator, the process is started, and the character information acquisition unit 21 acquires report character information relevant to the three-dimensional image including a plurality of tomographic images (step ST1). Then, the image character information conversion unit 22 converts each of the plurality of tomographic images into image character information corresponding to each tomographic image (step ST2).

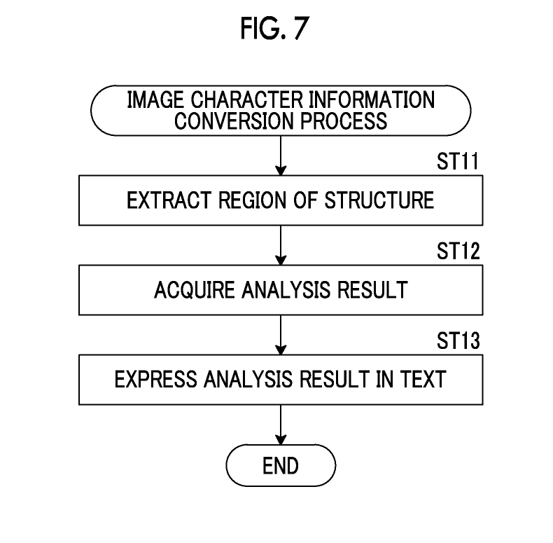

FIG. 7 is a flowchart of an image character information conversion process. In the image character information conversion process, the region extraction unit 30 extracts a region of at least one structure included in each tomographic image (step ST11). Then, the analysis unit 31 analyzes the region of the structure extracted from the tomographic image to acquire the analysis result on the disease and the like included in each tomographic image (step ST12), expresses the acquired analysis result in text (step ST13), and ends image character information conversion processing.

Subsequent to step ST2, the specifying unit 23 specifies at least one relevant tomographic image, which is relevant to the interpretation report, from the plurality of tomographic images by comparing the report character information R0 with the image character information GT1 to GT4 corresponding to the plurality of respective tomographic images (step ST3). Then, the display control unit 24 displays an interpretation report screen including the specified relevant tomographic image and the interpretation report on the display unit 14 (step ST4), and ends the process.

As described above, in the present embodiment, the report character information relevant to the three-dimensional image including a plurality of tomographic images is acquired, each of the plurality of tomographic images is converted into image character information corresponding to each tomographic image, and a relevant tomographic image is specified by comparing the report character information with the image character information corresponding to each of the plurality of tomographic images. Here, the processing for converting an image into characters can be performed at a higher speed than the processing for converting characters into an image. Therefore, according to the present embodiment, a tomographic image relevant to the report character information acquired from the interpretation report can be specified at high speed.

In the embodiment described above, the specifying unit 23 may specify two or more relevant tomographic images, which are relevant to the report character information, from the plurality of tomographic images. In this case, all of the tomographic images whose scores output from the discriminator 23A exceed the threshold value Th1 may be specified as relevant tomographic images. For example, assuming that the threshold value Th1 is 0.6, in a case where the scores of the tomographic images S1 to S4 are 0.2, 0.4, 0.8, and 0.7, the tomographic image S3 and the tomographic image S4 may be specified as relevant tomographic images that are relevant to the report character information.

In the embodiment described above, there is a possibility that a relevant tomographic image cannot be specified. In a case where a relevant tomographic image cannot be specified, the specifying unit 23 may output the result of “no relevant tomographic image”. In this case, the display control unit 24 may display “no relevant tomographic image” on the display unit 14.

In the embodiment described above, the analysis unit 31 of the image character information conversion unit 22 analyzes the tomographic image. However, an external analysis server or the like may analyze the tomographic image.

In the embodiment described above, the medical image group is a three-dimensional image that is a CT image, and the medical image is a tomographic image forming a three-dimensional image. However, the invention is not limited thereto. The medical image group may be a three-dimensional image that is an MRI image, and the medical image may be a tomographic image forming a three-dimensional image that is an MRI image.

In the embodiment described above, the character information relevant to the medical image group is acquired from the interpretation report. However, the character information relevant to the medical image group may be acquired from a medical document other than an interpretation report, such as an electronic medical record and a diagnosis report.

What is claimed is:

1. A medical image specifying apparatus, comprising:
a character information acquisition unit that acquires character information relevant to a medical image group including a plurality of medical images;
an image character information conversion unit that converts each of the plurality of medical images into image character information corresponding to each medical image; and
a specifying unit that specifies at least one relevant medical image, which is relevant to the character information, from the plurality of medical images by comparing the character information with the image character information corresponding to each of the plurality of medical images.

2. The medical image specifying apparatus according to claim 1,
wherein the image character information conversion unit comprises:
a region extraction unit that extracts a region of at least one structure included in each of the medical images; and
an analysis unit that analyzes the extracted region of the structure and expresses features of the region of the structure in text.

3. The medical image specifying apparatus according to claim 2,
wherein the features of the region of the structure include at least one of a position of a lesion included in the structure, a type of the lesion, or a size of the lesion.

4. The medical image specifying apparatus according to claim 1,
wherein the specifying unit further specifies a position relevant to the character information in the relevant medical image.

5. The medical image specifying apparatus according to claim 1, further comprising:
a display control unit that displays the specified relevant medical image and the character information on a display unit.

6. The medical image specifying apparatus according to claim 4, further comprising:
a display control unit that displays the specified relevant medical image and the character information on a display unit.

7. The medical image specifying apparatus according to claim 6,
wherein the display control unit highlights a position relevant to the character information in the relevant medical image.

8. The medical image specifying apparatus according to claim 1,
wherein the medical image is a tomographic image, and
the medical image group is a three-dimensional image including a plurality of the tomographic images.

9. A medical image specifying method, comprising:
acquiring character information relevant to a medical image group including a plurality of medical images;
converting each of the plurality of medical images into image character information corresponding to each medical image; and
specifying at least one relevant medical image, which is relevant to the character information, from the plurality of medical images by comparing the character information with the image character information corresponding to each of the plurality of medical images.

10. A non-transitory computer-readable storage medium that stores a medical image specifying program causing a computer to execute:
a step of acquiring character information relevant to a medical image group including a plurality of medical images;
a step of converting each of the plurality of medical images into image character information corresponding to each medical image; and
a step of specifying at least one relevant medical image, which is relevant to the character information, from the plurality of medical images by comparing the character information with the image character information corresponding to each of the plurality of medical images.
